(19) United States
(12) Patent Application Publication
(10) Pub. No.: US 2023/0181222 A1
(43) Pub. Date: Jun. 15, 2023

(54) ADJUSTABLE ROD

(71) Applicant: CTL Medical Corporation, Addison, TX (US)

(72) Inventors: Chad HARTLEY, Lake Havasu City, AZ (US); Jon SUH, Ambler, PA (US); Sean SUH, Milltown, NJ (US)

(21) Appl. No.: 18/081,095

(22) Filed: Dec. 14, 2022

Related U.S. Application Data
(60) Provisional application No. 63/289,567, filed on Dec. 14, 2021.

(51) Int. Cl. A61B 17/70

(52) U.S. Cl. CPC A61B 17/7029

(57) ABSTRACT

Disclosed are devices, systems and/or methods for use in the surgical treatment of vertebrae and/or other bones, in particular elongated systems and devices including flexible connecting members than can rigidified in a desired manner during a surgical procedure to provide corrective forces to the spine.

### CROSS-REFERENCE TO RELATED APPLICATIONS

This application claims the benefit of U.S. Provisional Application No. 63/289,567 entitled “Adjustable Rod” filed Dec. 14, 2021, the disclosure of which is incorporated by reference herein in its entirety.

### FIELD OF THE INVENTION

The present invention relates generally to the field of instrumentation, devices, and systems for treatment of the spine, and more particularly to methods and systems for interconnecting two or more portions of the spine and related devices using a flexible elongated member capable of selective rigidification.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and/or immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient’s pain and preventing neurological loss of function.

While the external stabilization systems of the prior art are a step in the right direction, there remains room for additional improvements. Oftentimes, the geometric and dimensional features of these systems and patient anatomy constrain the surgeon during surgery and prevent optimal placement, attachment and loading of the spinal motion segment. Thus, there is a general need in the industry to provide methods and devices for stabilizing a spinal motion segment that reduce surgeon constraints during surgery and optimize placement and attachment of the stabilization devices to the spinal motion segment. There is also a need for stabilization devices that maintain the desired corrective forces on the spinal motion segment. The present invention is directed to meeting these needs and others in a novel and unobvious manner.

### BRIEF SUMMARY OF THE INVENTION

In various disclosed embodiments, difficult mechanical fixation issues associated with implant and anatomical constraints in a given patient may be minimized and/or eliminated by using implantable devices such pedicle screws and related components in conjunction with one or more fixation rods that may be convertible between a flexible and a rigid state. These rods may include a proximal locking end, a plurality of mid-positioned flexible compression components and a distal pulling or locking base. In a flexible state, the rod may be easily manipulated using a surgeon’s hands or normal surgical tools to be shaped and conformed to a desired shape, such as a non-straight pathway, and in the rigid state the rod will resist the tensile, shear, rotational and/or compressive loads exerted on the rod by attached devices such as pedicle screws or other hardware.

Methods of use of the disclosed embodiments may include device conversion between flexible and rigid states using a proximal locking end and elated components.

In an embodiment, the flexible rod will include a plurality of individual segments having a mechanical engagement structure for non-rigidly interlocking the individual segments together. The segments can desirably include one or more textured and/or roughened surfaces, channels and/or apertures which are arranged to allow the segments to be slightly spaced apart, yet still remain mechanically linked) and the segments may be rotated and/or repositioned relative to each other in the flexible rod body when the segments are in the non-rigid mechanical engagement with each other. For example, the individual segments will desirably move relative to each other in a plurality of orthogonal planes relative to each other, which may include polyaxial and/or mono-axial movement between two adjacent individual segments. The flexible rod will desirably incorporate a compression member positioned on or in the proximal locking end which can draw the locking base towards the proximal locking end and desirably compress the individual segments therebetween, with the individual segments coming into intimate compressive contact with each other and frictional or other mechanical locking features acting between the individual segments to immobilize or “freeze” the locking segments in a current relative orientation, causing the rod to assume a fixed, generally inflexible shape, which desirably can be further reversable to allow further manipulation and/or bending of the flexible rod where desired. Once the fixation rod has been placed in a desired shape and/or position, the compression member can be manipulated to lock the fixation rod in a final permanent orientation and/or shape.

Described herein is a fixation rod with an adaptable or alterable shape capable of deployment while optionally having a flexible or non-rigid form and/or inflexible or rigid form prior to, during and/or after deployment. The fixation rod may selectively revert to a flexible or non-rigid form subsequent to deployment for either further adjustment or removal from the surgical space. The fixation rod with adaptable or alterable shape shall also simply be referred to herein as the device, support device or the apparatus.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIGS. 1A and 1B depict perspective views of one exemplary embodiment of a flexible rod constructed in accordance with various teaching of the present invention;

FIG. 2A depict an exploded view of the flexible rod of FIGS. 1A and 1B;

FIG. 2B depicts a cross-sectional view of a fully assembled fixation rod of FIG. 2A.

FIG. 3 depicts the flexible rod of FIGS. 1A and 1B during deformation;

FIGS. 4A and 4B depict views of one exemplary embodiment of a modular compression element or individual segment;

FIG. 4C depicts a cross-sectional view of the modular compression element of FIGS. 4A and 4B;

FIGS. 5A and 5B depict views of an upper locking end positioned at a proximal end of the flexible rod of FIGS. 1A and 1B;

FIG. 5C depicts a cross-sectional view of the upper locking end of FIGS. 5A and 5B;

FIGS. 6A and 6B depict views of a set screw with tapered tip;

FIGS. 7A and 7B depict views of a translation cam;

FIG. 8 depicts an axial connection rod;

FIGS. 9A and 9B depict views of a friction or securement pin;

FIGS. 10A and 10B depict views of a lower pulling end;

FIG. 11 depicts a partial cross-sectional view of an assembled upper locking end; and

FIG. 12 depicts a schematic view of the step of tensioning and rigidifying segments of a fixation rod portion.

### DETAILED DESCRIPTION OF THE INVENTION

For the purposes of promoting an understanding of the principles of the invention, reference will now be made to the embodiments illustrated in the drawings and specific language will be used to describe the same. It will nevertheless be understood that no limitation of the scope of the invention is hereby intended, such alterations and further modifications in the illustrated devices, and such further applications of the principles of the invention as illustrated herein being contemplated as would normally occur to one skilled in the art to which the invention relates.

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

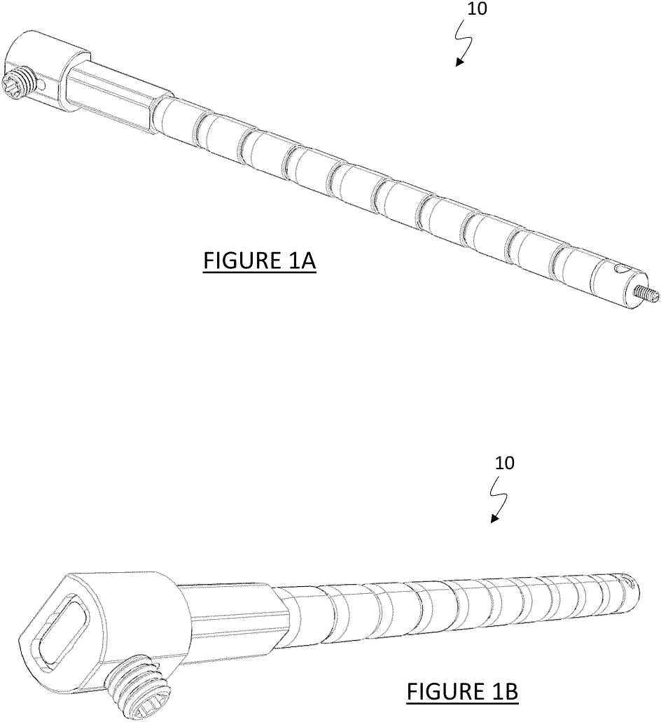

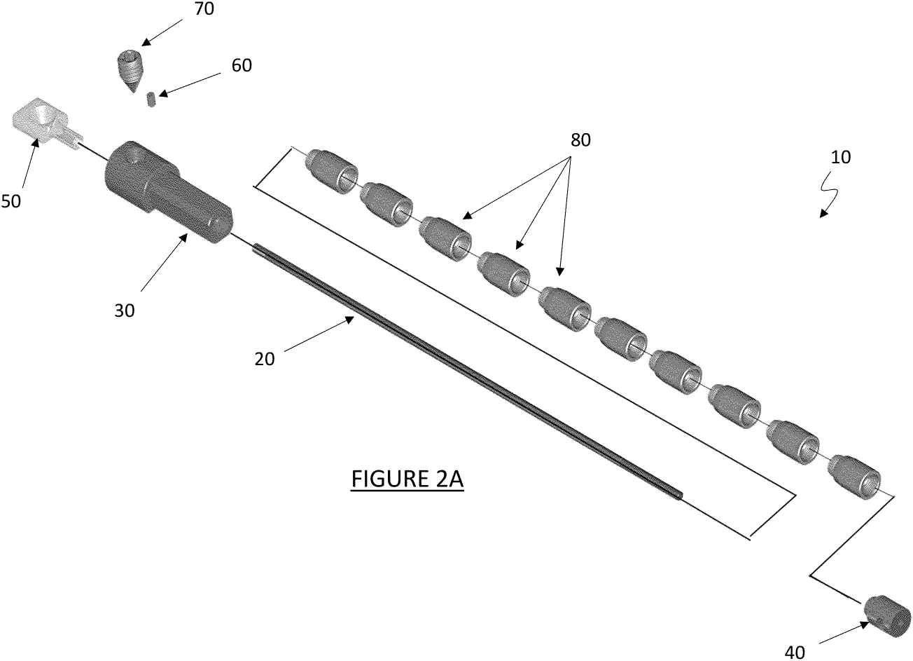

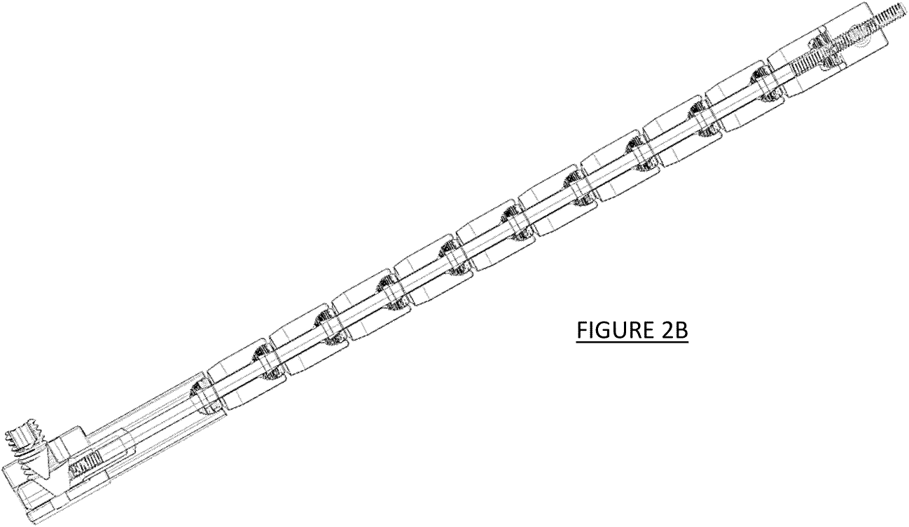

FIGS. 1A and 1B depict one exemplary embodiment of a flexible rod 10 which can be utilized in a variety of surgical procedures, such as a fixation rod for spinal fusion procedures. As best seen in the exploded view of FIG. 2A (and also the fully assembled cross-sectional view of FIG. 2B), the flexible rod can 10 include an axial connection or tension rod 20, an upper locking end 30, a lower pulling end 40, a translation cam 50, a deformable friction pin 60, a cam translating set screw 70 and a plurality of modular compression elements 80. In use, the axial connection rod is secured to the upper locking end 30 and the lower pulling end 40, with a plurality of the modular compression elements 80 positioned therebetween.

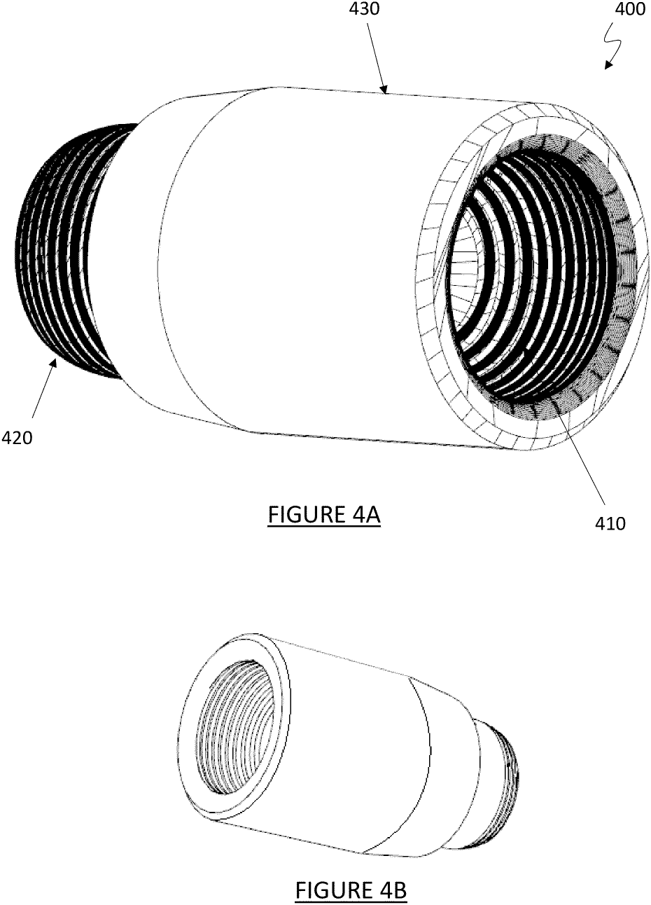

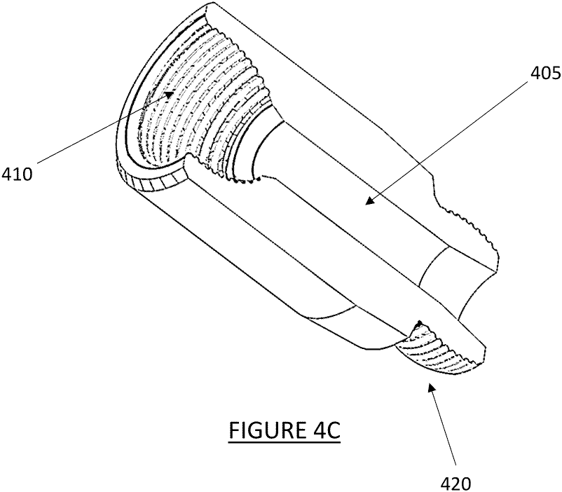

FIGS. 4A through 4C depict views of one exemplary embodiment of a modular compression element or individual segment 400. The flexible rod desirably includes a plurality of individual segments 400, with the exemplary embodiment of FIG. 1A depicting 10 segments, with each segment having a central longitudinally extending lumen 405 therethrough (see FIG. 4C), and may further incorporate frictional and/or mechanical engagement structures for non-rigidly interlocking respective individual segments together. Each of the segments can include a proximal female end 410 and a distal male protrusion end 420, with each of these ends desirably including surface features 430 such as roughening, teeth, pyramidal protrusions, channels and/or or apertures arranged to generally hold the complimentary surfaces to each other when the segments 400 are compressed together. Desirably, a central connection rod 20 (see FIG. 2) extends through the central longitudinally extending lumen 405 of the segments 400, holding the segments in non-rigid loose frictional/mechanical engagement, but allowing the segments to slide longitudinally along the rod as desired (and limited by the presence of multiple segments). The individual segments 400 may move relative to the central connection rod to a certain limited degree, as the rod may approximate the size of the lumen 405 or may be substantially smaller than the lumen if desired (and a single rod may be replaced by multiple rods, if desired). In the disclosed embodiment, an outer surface 430 of the segment 400 can desirably be formed in a generally cylindrical size and/or shape, similar to the dimensions of existing surgical spinal fixation rod, which can allow for the flexible rod to be utilized with commercially available standard surgical screws and related spinal fixation system components.

While the disclosed embodiment depicts a generally cylindrical outer surface for each of the segments, it should be understood that other shapes and/or configurations for one or more of the segments could utilized with varying utility, including flattened outer surfaces, roughened or non-rounded shapes and/or regular or irregular polyhedral shapes (which may provide increased frictional and/or mechanical engagement between the engaging surfaces, as well various combinations of frictional/mechanical engagement thereof depending upon surface feature design and/or arrangement). In a similar manner, the individual segments could comprise modular components of the same or differing sizes and/or shapes, including individual segments of reduced width and/or diameter which could accommodate connection mechanisms or varying shapes and/or diameters.

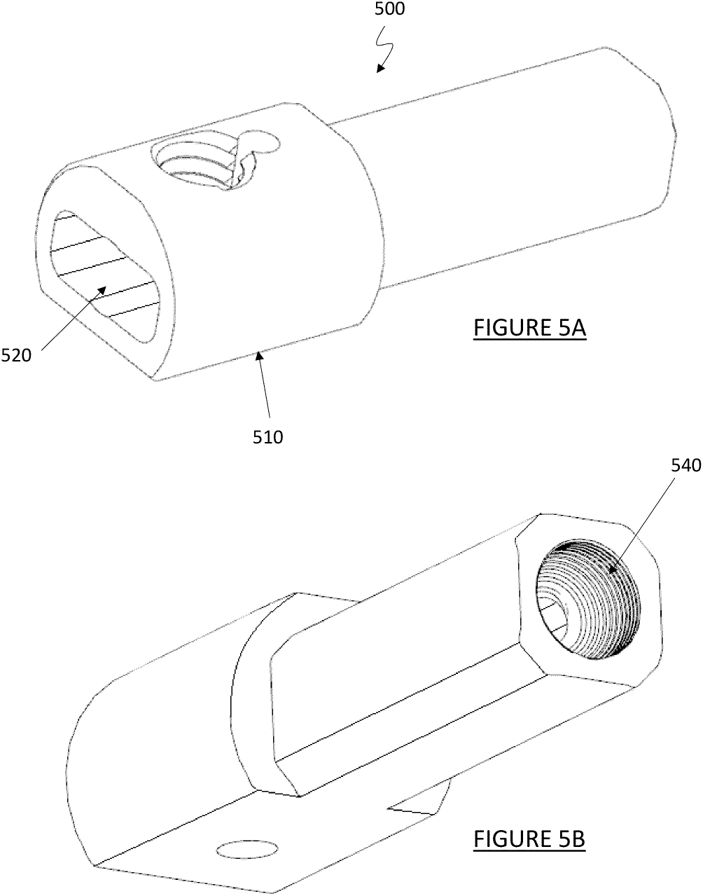

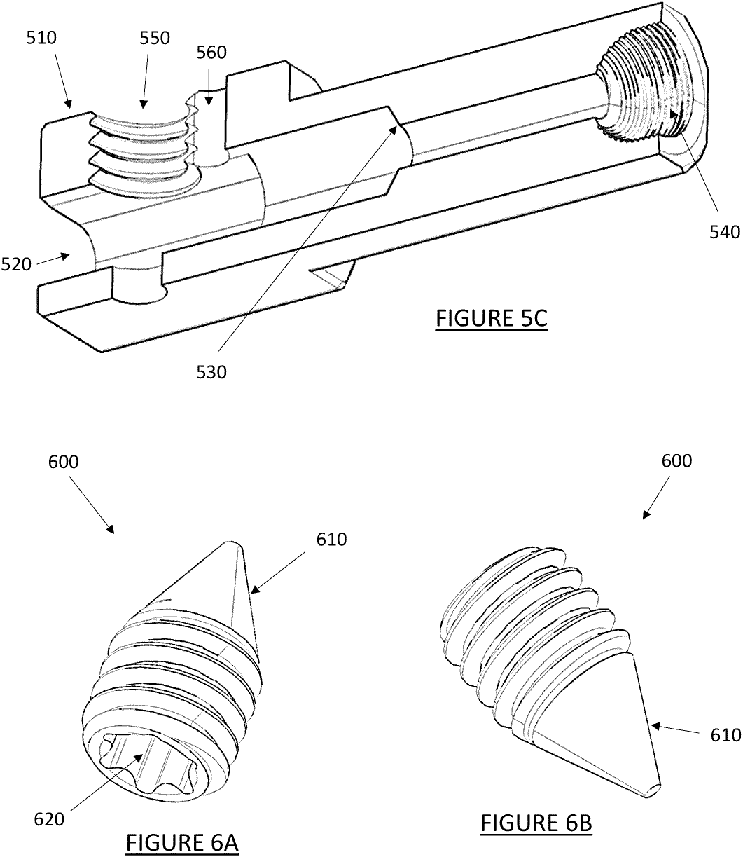

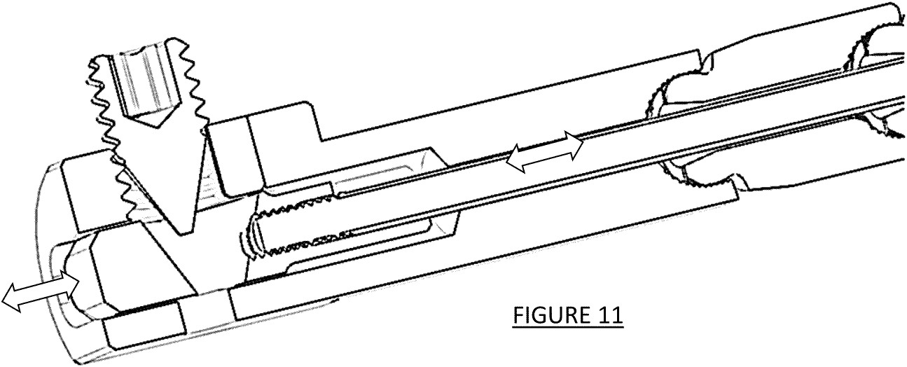

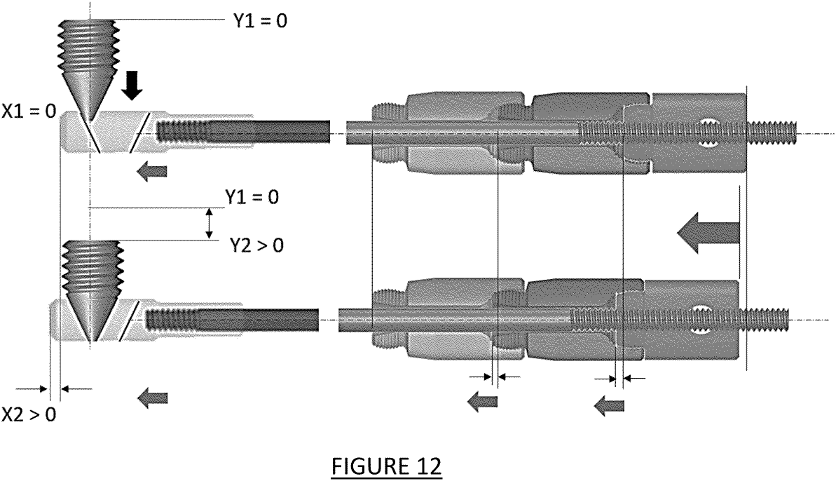

FIGS. 5A through 5C depict various views of an upper locking end 500, which desirably is positioned at a proximal end of the device 10. The upper locking end 500 includes a locking body 510 with a body opening 520 formed therein, the body opening 520 including a shoulder section 530 and a female end 540. The body opening 520 further includes an internally threaded set screw opening 550, and a friction pin opening 560. In use, a translation cam 700 can be positioned within the body opening 520, with a central connection rod secured to a distal opening 710 in the translation cam 700. A friction pin 900 is positioned in the friction pin opening 560 to retain the translation cam 700 within the body opening 520, with a set screw 600 threaded into the set screw opening 550. As best seen in FIGS. 11 and 12, advancement of the set screw 600 into the set screw opening 550 will desirably urge the translation cam 700 in a proximal direction (e.g., drawing tension on the rod and associated lower pulling end), desirably drawing on the central connection rod and associated lower pulling end to thereby compress the various individual segments together and engaging the surface features to mechanical lock the segments relative to each other and relative to the upper locking end and lower pulling end.

FIGS. 6A and 6B depict views of an exemplary set screw 600 with a tapered tip 610, wherein the tapered tip 610 desirably engages with an angled surface on the translation cam, as previously described. The set screw 600 can also include a driving feature 620 such as a hexalobular or Torx™ recess, as is well known in the art.

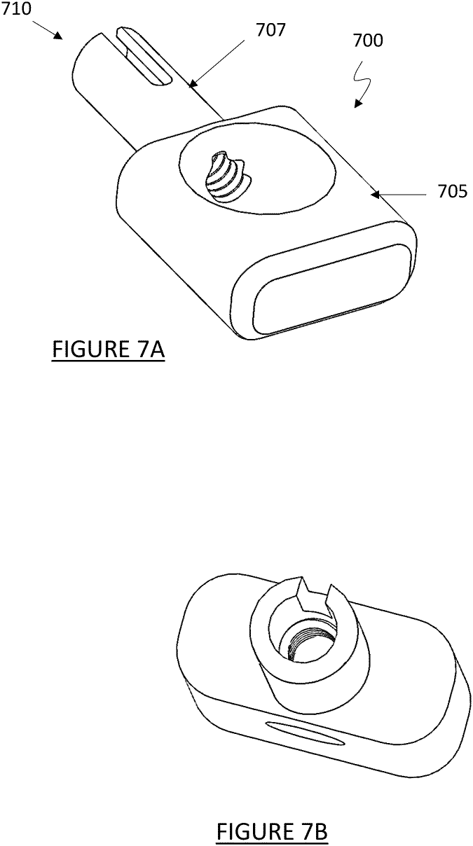

FIGS. 7A and 7B depict views depict views of an exemplary translation cam 700. As previously noted, the translation cam 700 can include a cam body 705 and protruding tube 707, with a distal opening 710 formed in the cam body which extends out through the tube 707. In the disclosed embodiment, at least a portion of the tube 707 can be internally threaded, to desirably mate with and retain a first rod end 810 of portion of a central connection rod 800.

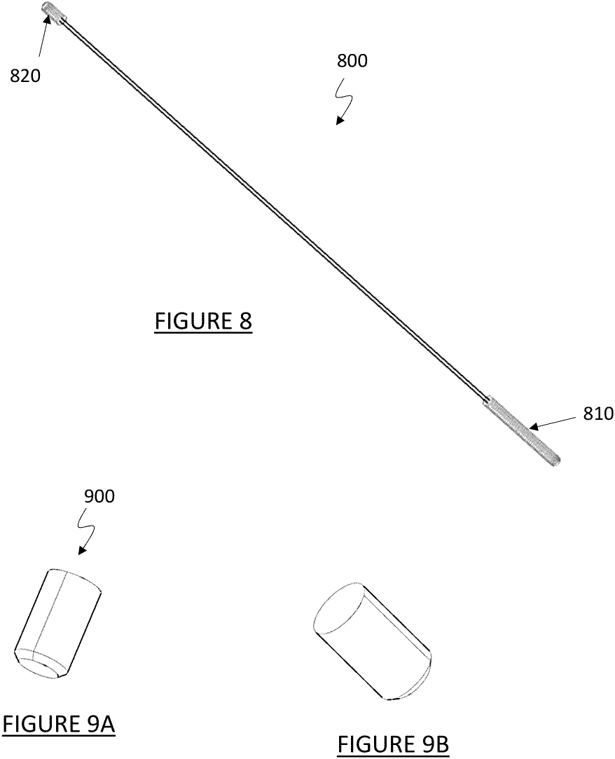

FIG. 8 depicts one embodiment of an axial connection or tension rod 800, which desirably is attached at a first end 820 to the translation cam 700 and at a second end 810 to a lower pulling end 1000. Desirably the rod 800 will extend through the various individual segments, as previously described. The rod 800 can be constructed of virtually any biocompatible material capable of applying a necessary tension to the locked device components, although generally ductile materials may be preferred such that a lesser level of force can be used to bend the rod and associated components of the flexible rod system to a desired “unlocked” shape, but which allows the flexible rod to maintain a bent shape once such lesser level of force application is halted without requiring locking of the flexible rod components.

FIGS. 9A and 9B depict views of a friction or securement pin 900, which can be used to retain the translation cam within the upper locking end (and desirably retain the various device components in an assembled, yet unlocked condition), yet allow the translation cam to move longitudinally to lock and unlock the individual segments upon advancement and/or withdrawal of the set screw as previously described.

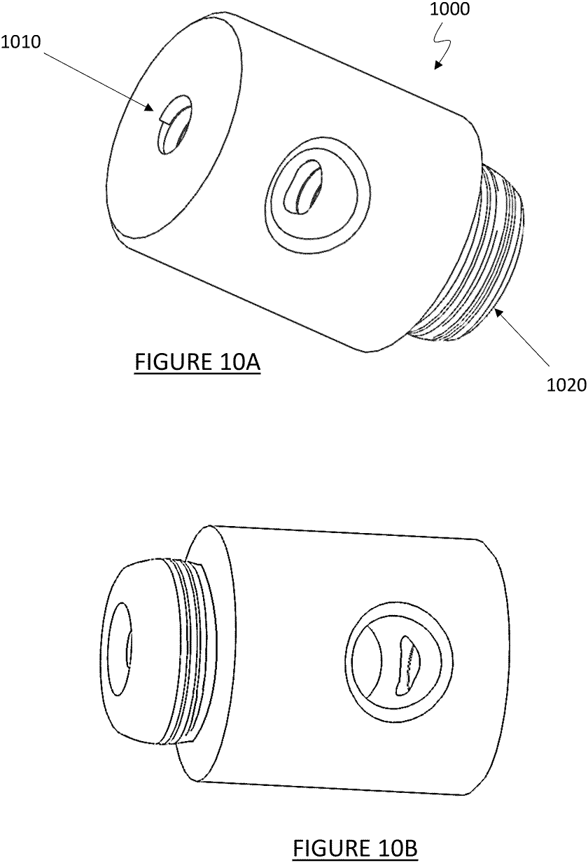

FIGS. 10A and 10B depict views of a lower pulling end 1000 which desirably includes a lower central bore 1010 which included an internally threaded portion that engages with the second end 810 of the central connection rod 800. The lower pulling end 1000 also includes a male protrusion end 1020, which desirably engages with a proximal female end 410 of an individual segment upon tensioning of the connection rod 800.

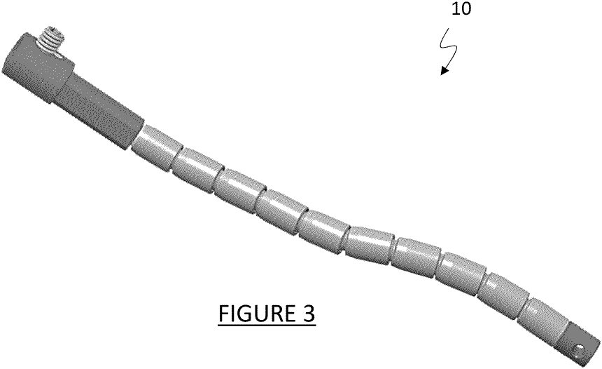

AS best seen in FIGS. 11 and 12, when the axial connection rod is not tensioned, the modular compression elements can be slightly spaced apart and/or not frictionally engaged with each other (see FIG. 12) and desirably capable of being repositioned relative to each other (with the underlying axial connection rod desirably deforming as necessary). However, when a tension force is drawn on the upper locking end by advancement of the set screw (see FIG. 12), the modular compression elements are desirably drawn towards each other, with contoured, rounded, curved and/or hemispherical engagement surfaces on each modular compression element desirably engaging and “locking” the modular compression elements in a desired relationship and position. This arrangement allows the rod to be manipulated into a variety of shapes, such as the complex curved shape depicted in FIG. 3. Because this embodiment of a non-tensioned flexible rod and underlying axial connection rod can be easily bent using normal hand pressure, but can then be tensioned to a substantially non-deformable final tensioned shape, the present invention can significantly simplify a given surgical procedure, and also obviate the need for bulky and expensive rod bending and cutting apparatus heretofore required in the operating room.

In various embodiments the fixation rod may be formed from a group of individual segments arranged in an end-to-end fashion, where each segment has a generally standardized shape. In various embodiments, the device may be flexible in more than two planes orthogonal to the general axis, although other embodiments flexible in only one or two planes orthogonal to the general axis may be useful in various stances. In some embodiments the standardized shape of each segment may include a generally cylindrical body having a first end and a second end. The first end may have a protrusion extending from the cylindrical body, and that protrusion may be formed or shaped so as to enter and/or engage with an aperture or receptacle shaped generally to receive the protrusion in a generally male to female arrangement. Each cylindrical body may have one male end and one female end at the first or second ends. Alternatively, the cylindrical body may have two male ends or two female ends (not shown). When the cylindrical bodies are lined up to form the device, the arrangement can be made so male ends are adjacent to and engage into female ends. The male and female connection and related components may be such that the adjacent segments will do not fully separate from each other (e.g., at least a portion of a male end may still remain within an adjacent female cavity) when the device is in a flexible (e.g., unlocked) configuration.

The number of individual segments in a flexible rod embodiment may vary, although in most embodiments a plurality of such segments would be incorporated into a single rod. For example, rods having 2, 3, 4, 5, 6, 7, 8, 9, 10, 1, 12, 13, 14, 15, 16, 17, 18, 19, or 20 or more segments are contemplated herein. If desired, a flexible rod design may allow for addition or removal of segments to increase or decrease rod length as desired.

In various embodiments, the connection rod may be replaced with a flexible fiber or similar construct which provides tension for locking of the individual segments, but which does not provide the limited rigidi of the connection rod (which may allow the flexible rod to deform under its own weight when in an unlocked condition). Such alternative embodiments may prove useful in a variety of surgical situations, and such construction is contemplated herein.

In various embodiments, the flexible rod may include a series of cylindrical or other shaped bodies arranged in an end-to-end fashion, with a male to female mechanical arrangement there between. In at least one embodiment, the body segments may have at least one centrally positioned lumen extending therethrough running parallel to the general longitudinal axis of the device. The hole(s) may form one or more lumens in the device when the segments are generally lined up, with at least one rod or fiber extending through the hole(s) in the segments, such that the rod/fiber extend from one end of the device to the other through the hole(s) in the segments. In various embodiments the holes will desirably line up to form a lumen through the device.

In various embodiments, the rod/fiber extending through the holes can be affixed to the most distal component and extend into the most proximal component so as to engage a tapered locking screw and interface. The tapered locking screw and interface are configured to tension the rod/fibers that extend to the distal lock, thereby changing the construct from a flexible state to a rigid state or vice versa. In various embodiments, the rod/fibers may be manipulated to fix the segments into a rigid or inflexible straight, curved and/or composite shape. Desirably, loosening of the rod/fibers may subsequently return the device into a flexible or non-rigid shape. In various embodiments, the rod/fibers may be advanced distally or retracted proximally while the device is either flexible or rigid. In some embodiments, the rod/fibers may be cables, wires, rods or similar structures. The fibers may be made of biocompatible metal (for example, stainless steel, titanium or nitinol), alloys, polymers, non-resorbable materials, ceramics, glass, carbon fiber or any combination of these materials, as well as other materials known in the art.

In various embodiments, the shape of the device can be facilitated by the central rod or wire which extends through a central aperture or hollow core of the device components. The central rod may initially have a particular geometry to facilitate creating a desired shape or path (e.g., a straight central rod which creates a generally straight yet flexible fixation rod). Desirably, the central rod can be easily bent using hand pressure of a surgeon and/or manipulation by standard surgical tools, with the device maintaining a desired curvature during and after deployment. The central rod may be tensioned to lock the individual segments in place relative to each other once a desired shape is acquired. The central rod may be a flexible or stiff wire, but will desirably be sufficiently flexible to allow alteration of the shape of the unlocked device as desired.

In some embodiments, the device may also have a polymer sleeve to help provide an atraumatic surface or other features between the fixation device and the surrounding anatomy and/or bone.

In accordance with various aspects of the present subject matter, the present invention provides a surgical fixation system with components interconnected by one or more flexible elongate members, wherein said flexible member(s) can be rigidified after assumption of a desired shape and/or size. Desirably, coupling members can be used to secure the elongate member to the anchors to maintain the corrective forces applied to the spinal motion segment by the elongate member. Desirably, the flexible elongate member can be rigidified at any desired point during the surgical procedure, which could include the initiation and/or reversal of rigidification at multiple times during a given procedure, including where the surgeon may wish to utilize the rigid elongated rod to manipulate one or more connected vertebrae (possibly in conjunction with subsequent restoration of flexibility for other surgical purposes after the desired spinal manipulation is completed).

In some embodiments, the flexible elongate member can be altered to assume a desired shape and then rigidified prior to attachment to spinal anchors, while in other embodiments the flexible elongate member can be attached to one or more spinal anchors and then rigidified to match the natural contours of the patient’s spine. In still other embodiments, a flexible elongated member may include a plurality of independently actuable flexible sections, wherein each section or portions thereof may be capable of being rigidified or reversed back to a flexible status without affecting adjacent areas of the member.

It should be understood that the various system components may be utilized in all regions of the spine, including the cervical, thoracic, lumbar, lumbo-sacral and sacral regions of the spine. It should also be understood that the system components may extend across a spinal motion segment having only two vertebrae or more than two vertebrae. It is further contemplated that two or more flexible elongate members may be employed simultaneously along the same spinal motion segment. Additionally, although system components may be shown as having application in a posterior region of the spinal motion segment, similar components may alternatively be applied in other surgical approaches and combinations of surgical approaches to the spinal motion segment such that one or more stabilization systems are attached to the anterior, antero-lateral, lateral, and/or postero-lateral portions of the spinal motion segment.

A stabilization system including the disclosed flexible elongate members can allow at least a small degrees of motion between each of the individual components of the member (when such flexion is allowed), ultimately resulting in a relatively large degree of deformation for the member over a plurality of such segments. It should be understood that the disclosed stabilization system could be used in conjunction with fusion or non-fusion treatment of the spine.

In the various embodiments described herein, the flexible rod may be composed from a polymer, a metal, an alloy, or a combination thereof, which may be biocompatible. For example, the stabilization device can be formed from titanium or a titanium alloy. Other suitable metals may include stainless steel, cobalt-chromium alloys, and tantalum. In some embodiments, metal alloys having shape memory capability, such as nickel titanium or spring stainless steel alloys, may also be used. In some embodiments, the stabilization device can be formed from a suitable polymer including non-degradable polymers, such as polyetheretherketone (PEEK) and polyethylene (PE), as well as modified versions of these materials (for example, PEEK+calcium phosphates and PE+vitamin E, metal coatings, or surface texturing). Additional non limiting polymers may include; polyether-block co-polyamide polymers, copolyester elastomers, thermoset polymers, polyolefins (e.g., polypropylene or polyethylene, including high density polyethylene (HDPEs), low-density polyethylene (LDPEs), and ultrahigh molecular weight polyethylene (UHMWPE)), polytetrafluoroethylene, ethylene vinyl acetate, polyamides, polyimides, polyurethanes, polyvinyl chloride (PVC), fluoropolymers (e.g., fluorinated ethylene propylene, perfluoroalkoxy (PEA) polymer, polyvinylidene fluoride, etc.), polyetheretherketones (PEEKs), PEEK-carbon fiber composites, polyetherketoneketones (PEKKs), poly(methylmethacrylate) (PMMA), polysulfone (PSU), epoxy resins, and silicones.

In one form, a flexible elongate member can comprise one or more components comprising metallic materials such as stainless steel, titanium, a titanium alloy or chrome cobalt, one or polymers, such as, for example, polyester or polyethylene, ultra-high molecular weight polyethylene (UHWMPE) or Polyether Ether Ketone (PEEK); one or more superelastic metals or alloys, such as, for example, nitinol; or from resorbable synthetic materials, such as, for example suture material or polylactic acid. In some embodiments, the member may include components having elasticity such that the member may return toward a predefined alignment and/or state when rigidification may be released and/or reversed.

Additional materials used with the disclosed embodiments may include carbon and polyaramid structures, glass or fiberglass derivatives, ceramic materials, and artificial biocompatible protein derivatives (recombinant derived collagen). In other embodiments, the various components of the flexible rod may be made of a metal and/or alloy segments with a polymer shell, or a sandwich style and coaxial extrusion composition of any number of layers of any of the materials listed herein. Various layers may be bonded to each other to provide for single layer composition of metal(s), alloys, and/or polymers. In another embodiment, a polymer core may be used with a metal and/or metal alloy shell, such as a wire or ribbon braid.

If desired, at least a portion of the flexible rod may be treated or coated with a silicon nitride material, or a calcium material, such as calcium deposits, calcium phosphate coatings, calcium sulfates, modified calcium salts such as magnesium, strontium and/or silicon substituted calcium phosphates, RGD sequences, collagen, and combinations thereof in order to enhance a strength of bone ingrowth, on-growth, and/or through-growth between the segments or other portions of the device.

The disclosed flexible elongate members and related system components described herein may be employed with a spinal fusion stabilization system, including in conjunction with interbody fusion devices. In addition, it contemplated that the components described herein may be employed in isolation or in systems that include two or more flexible elongate members and associated anchoring devices. Examples of other systems include: one or more elongate members extending laterally across a vertebral body; one or more elongate members extending in the anterior-posterior directions across a vertebral body; one or more elongate member wrapped around a vertebral body; and/or various combinations thereof.

In view of the systems and devices discussed above, a method for stabilizing a spinal motion segment will be described. One or more flexible elongate members can be provided with a length sufficient to extend along the spinal motion segment to which it is to be attached. Bone screws or other surgical anchors can be engaged to respective vertebrae comprising the spinal motion segment. Adjacent anchors can be manipulated, distracted and/or compressed toward one another to achieve a desired spinal correction, and the flexible elongate member attached to each of the anchors to provide corrective forces to the spinal motion segment. It is contemplated that the flexible elongate member can be rigidified before, during and/or after attachment to one or more anchors as discussed above. Since the anchors can attach to virtually any location along the length of the elongate member, the member need not include any holes, slots or other engaging features along its length for engagement to the anchors (unless such features are desired by the surgeon for a variety of reasons). Thus, a surgeon is not constrained to particular, discrete attachment locations along the elongate member, but rather is provided an infinite number of attachment locations along the elongate member.

If desired, the disclosed devices and/or components thereof can be constructed from a variety of modular components, including modular components comprising different materials. If desired, such modular components could be provided in a kit form for selection and/or assembly in a surgical theatre and/or in situ during a surgical procedure. If desired, various components may be removable and replaceable.

In some instances, it becomes necessary to remove a bone fixation device from a patient. The device of the present disclosure may be removed following a series of steps similar to, but not necessarily opposite of the implanting steps. In some embodiments, the device may be modified in-situ and/or removed from a patient’s anatomy by exposing the locking component section, returning the flexibility to the fixation rod, and altering or removing the device from the bone (which may include reactivating the locking component to rigidify the flexible rod.

In accordance with another aspect of the present subject matter, various methods for manufacturing devices and/or components thereof, as set for within any of the details described with the present application, are provided.

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, like reference numerals may represent similar parts throughout several views of the drawings.

The terms “including,” “comprising” and variations thereof, as used in this disclosure, mean “including, but not limited to,” unless expressly specified otherwise. The terms “a,” “an,” and “the,” as used in this disclosure, mean “one or more,” unless expressly specified otherwise.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

The present invention provides various devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention.

As previously noted, the various embodiments of an implant disclosed herein can be configured to interact with two bone vertebrae of a spine or other anatomical locations. The spine may have any of several types of spinal curvature disorders which are sought to be treated. Examples of such spinal curvature disorders include, but need not be limited to, lordosis, kyphosis, scoliosis and/or low and/or high velocity fractures, among other pathologies.

In various exemplary scenarios, a variety of surgical tools can be used in conjunction with various implant devices utilized to fix and/or secure adjacent vertebrae that have had cartilaginous disc between the vertebrae replaced with fusion material that promotes the fusion of the vertebrae, such as a graft of bone tissue. Also, such can be accomplished even when dealing with a spinal curvature disorder (e.g., lordosis, kyphosis and scoliosis).

Of course, method(s) for manufacturing the various devices and related components and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An elongated flexible member for implanting across two or more vertebrae, comprising:
a plurality of individual flexible segments, each flexible segment including a body having a first end and a second, with a lumen extending through the body from the first end to the second end, the first end of each body including an enlarged section having an internally roughened inner surface, the second end of the each body including an externally roughened outer surface portion,
an elongated semi-rigid rod member having an outer diameter that is less than an inner diameter of the lumens of the plurality of individual flexible segments, the plurality of individual flexible segments positioned over the elongated semi-rigid rod member in a longitudinally spaced apart configuration,
wherein, when the plurality of individual flexible segments are in a first, loosened configuration they can freely rotate relative to each other, but when the plurality of individual flexible segments are in a second, tightened configuration the individual flexible segments form an elongated rigid member.

2. The elongated flexible member of claim 1, wherein the plurality of individual flexible segments are in the second, tightened configuration when a tension force is applied to the elongated semi-rigid rod member.

3. The elongated flexible member of claim 1, wherein the elongated semi-rigid rod member has a first proximal rod end and a second distal rod end, and the plurality of individual flexible segments are positioned between the first and second rod ends.

4. The elongated flexible member of claim 1, wherein the first end of each of the individual flexible segments is positioned towards the first proximal rod end.

5. The elongated flexible member of claim 1, wherein the second end of each of the individual flexible segments is positioned towards the second distal rod end.

6. The elongated flexible member of claim 1, wherein the second end of at least one of the plurality of individual flexible segments is positioned within a first end of an adjacent one of the plurality of individual flexible segments.

7. The elongated flexible member of claim 2, wherein the plurality of individual flexible segments are frictionally locked together.

8. The elongated flexible member of claim 2, wherein the plurality of individual flexible segments are mechanically locked together.

9. The elongated flexible member of claim 1, wherein advancement of a set screw causes the plurality of individual flexible segments to assume the second, tightened configuration, and withdrawal of the set screw causes the plurality of individual flexible segments to assume the first, loosened configuration.

10. The elongated flexible member of claim 1, wherein at least a portion of the elongated flexible member comprises a bony ingrowth surface.

11. A medical apparatus for bone fixation, comprising:
a flexible body, including
a proximal end,
a distal end,
a tension rod extending between the proximal and distal ends, and
a plurality of individual lockable segments extending between the proximal end and the distal end, each of the plurality of individual lockable segments having a protrusion extending from a first end and a concave portion extending into a second end of the, with a longitudinally extending lumen extending from the first end to the second end, wherein the flexible tension rod extends through the longitudinally extending lumens of each of the plurality of individual lockable segments,
wherein, when the flexible tension rod is in a first, non-tensioned position, the individual locking segments can be displaced to a plurality of relative positions relative to each other and, when the flexible tension rod is in a second, tensioned position, the individual locking segments cannot be displaced relative to each other.

12. The medical apparatus for bone fixation of claim 11, wherein the tension rod comprises a semi-rigid rod.

13. The medical apparatus for bone fixation of claim 11, wherein the tension rod comprises a flexible rod.

14. The medical apparatus for bone fixation of claim 11, wherein the tension rod comprises a flexible fiber.

15. The medical apparatus for bone fixation of claim 11, further comprising a bony ingrowth surface on at least a portion of one of the plurality of individual lockable segments.

16. The medical apparatus for bone fixation of claim 11, wherein the flexible tension rod can be reversed from the second tensioned position to the first, non-tensioned position.

17. The medical apparatus for bone fixation of claim 11, wherein the tension rod can be bent using normal average hand pressure in the first, non-tensioned position, but cannot be bent using normal average hand pressure in the second, tensioned position.

18. The medical apparatus for bone fixation of claim 11, wherein a longitudinal length of the flexible body can be altered by adding or removing one or more of the plurality of individual lockable segments.

19. The medical apparatus for bone fixation of claim 11, wherein the flexible body has a first longitudinal length in the first, non-tensioned position and a second longitudinal length in the second, tensioned position, and the first longitudinal length is longer than the second longitudinal length.

20. The medical apparatus for bone fixation of claim 11, wherein each of the plurality of individual lockable segments are of a similar size and shape.

21. (canceled)
